(19) United States
(12) Patent Application Publication
(10) Pub. No.: US 2020/0138359 A1
(43) Pub. Date: May 7, 2020

(54) MEDICAL IMAGING

(71) Applicant: Oxford University Innovation Limited, Oxford (GB)

(72) Inventors: Michael PAVLIDES, Heading Oxford (Oxfordshire) (GB); Stefan NEUBAUER, Heading Oxford (Oxfordshire) (GB); Elizabeth TUNNICLIFFE, Heading Oxford (Oxfordshire) (GB); Eleanor BARNES, Oxford (Oxfordshire) (GB); Rajarshi BANERJEE, Oxford (Oxfordshire) (GB)

(21) Appl. No.: 16/734,445

(22) Filed: Jan. 6, 2020

Related U.S. Application Data
(63) Continuation of application No. 15/302,693, filed on Oct. 7, 2016.

(30) Foreign Application Priority Data
Apr. 8, 2014 (GB) 1406304.4

(51) Int. Cl. A61B 5/00; G06T 7/00; G01R 33/50; A61B 5/055; A61B 5/021

(52) U.S. Cl. CPC A61B 5/4244; A61B 5/7278; G06T 7/0014; G01R 33/50; A61B 5/416; G06T 2207/30101; A61B 5/021; G06T 2207/30104; G06T 2207/30048; G06T 2207/30056; G06T 2207/10088; A61B 5/055

(57) ABSTRACT

The present invention relates to methods for assessing or obtaining an indication of vascular pressure associated with organs or visceral tissues of the body by using MRI imaging methods. The invention particularly relates to methods for assessing or obtaining an indication of portal hypertension using Magnetic Resonance T1, or T1 and T2* relaxometry, and T1, T2 and/or T2* mapping of the liver or spleen.

### CROSS-REFERENCE TO RELATED APPLICATIONS

This application is a Continuation of U.S. patent application Ser. No. 15/302,693, filed Oct. 7, 2016, which is the National Stage of International Application No. PCT/GB2015/051064, filed 7 Apr. 2015, which claims the benefit of and priority to GB application 1406304.4, filed 8 Apr. 2014, the contents of all of which are incorporated by reference as if fully set forth herein.

### TECHNICAL FIELD

The present invention relates to methods for assessing or obtaining an indication of vascular pressure associated with organs or visceral tissues of the body by using MRI imaging methods. The invention particularly relates to methods for assessing or obtaining an indication of portal hypertension using Magnetic Resonance T1, or T1 and T2* relaxometry, and T1, T2, and/or T2* mapping of the liver or spleen.

### BACKGROUND

The liver is the largest internal organ and it plays a crucial role in many bodily functions such as metabolic homeostasis, nutrition, detoxification and blood clotting. It is characterised by the ability to regenerate, which allows it to deal with acute insults (e.g. acute viral infections) without any long term after-effects.

The liver has a dual blood supply. It receives arterial blood through the hepatic artery but also receives blood from the portal vein. The portal vein is formed from the mesenteric veins that drain the gut and the splenic vein that drains the spleen. Venous blood drains from the liver via the hepatic veins to the inferior vena cava and back to the heart.

Whilst the liver has the capability to regenerate itself, if it is subjected to repeated insults (e.g. inflammation, vascular congestion, etc.), it begins to accumulate scarring (fibrosis). Liver inflammation can be caused by a variety of conditions including alcohol abuse and chronic viral hepatitis, such as hepatitis C. Vascular congestion in the liver can be caused if there are blockages in the hepatic veins that drain blood from the liver or anything that leads to increases in the central venous pressure (e.g. right heart failure or congestive heart failure). If liver inflammation or blood congestion remains unchecked, over time severe scarring (cirrhosis) can develop.

Liver fibrosis is the hallmark of liver disease and typically accumulates over decades. In a patient, liver fibrosis may be suspected based on the clinical history (e.g. alcohol abuse) or abnormal liver blood tests. However, symptoms of liver disease are often not apparent until the disease reaches an advanced stage.

The gold standard for assessing liver fibrosis is a liver biopsy (Bravo et al. (2001)). To monitor how liver fibrosis progresses, patients will typically have repeated biopsies every 4-5 years. Liver fibrosis is assessed on these histological samples using numerical scales such as the Ishak score (Ishak et al. (1995)) which ranges from 0 (no fibrosis) to 6 (cirrhosis, severe scarring). Whilst biopsies can provide a good measure of the level of liver fibrosis (at less than an Ishak score of 6), biopsies do not accurately measure the severity of cirrhosis and so are of limited value in patients with liver cirrhosis. Additionally, liver biopsies are highly invasive procedures and only sample a tiny fraction of the whole liver, and so they cannot always give an accurate assessment of the state of the whole liver. Even after cirrhosis is established in the liver, scarring continues to accumulate and the patients are at risk of developing complications like liver cancer and portal hypertension.

Severe liver fibrosis (cirrhosis) can lead to impairment in the flow of blood through the liver (increasing hepatic vascular resistance). This can lead to the build-up of pressure in the portal vein (the blood vessel that drains blood from the gut and spleen).

Portal hypertension is a state of increased pressure in the blood vessels of the gut, liver and spleen. In the context of liver disease, the degree of portal hypertension reflects the severity of fibrosis. Portal hypertension is the underlying cause for the complications of liver cirrhosis and is the reason that patients with liver cirrhosis develop life-threatening complications, such as gastrointestinal bleeding.

Therefore, differentiating those patients with portal hypertension from those without is an important aspect of the care of patients with liver cirrhosis. The presence and the degree of portal hypertension are also important in determining the prognosis of patients; those with higher portal pressure are more at risk of complications and mortality (Merkel and Montagnese (2011)).

The measurement of portal pressure is therefore an important aspect in the assessment of patients with liver disease, because it gives an indication of the overall severity of liver disease (as opposed to a liver biopsy which only assesses a minute fraction of the liver). Furthermore, the portal pressure measurements are made using a continuous numerical scale (similar to blood pressure measurements) as opposed to the categorical scores used to assess liver biopsies. Portal pressure measurements can therefore detect small changes in the severity of liver disease that would otherwise be undetected.

Evaluation of portal pressures usually relies on an invasive technique, where a vascular catheter is inserted through a large peripheral vein (e.g. the internal jugular vein in the neck or the antecubital vein in the arm) and under X-ray guidance is advanced to the superior vena cava and then through the right atrium of the heart down to the liver. Before any measurements are taken, the system is calibrated to the pressure in the peripheral vein which is used as the reference against which all subsequent pressure measurements are made. Once in the liver vasculature, the catheter is advanced further until it gets “wedged” and a pressure measurement is taken using a transducer (i.e. the Hepatic Wedge Pressure, HWP). The catheter is then withdrawn until it lies free in the Hepatic Vein and a pressure measurement is taken in this position (i.e. Free Hepatic Vein Pressure, FHVP). The difference between the Hepatic Wedge Pressure and the Free Hepatic Vein Pressure is the Hepatic Vein Pressure Gradient (HVPG). The HVPG and HWP are frequently used as the closest available approximations to the true portal pressure (Bosch et al. (2009) and Escorsell of al. (1999))

In the West, liver cirrhosis is the commonest cause of portal hypertension. There are, however, other conditions that can lead to pathological increases in portal pressure.

These are usually categorised into conditions arising before the liver (pre-hepatic), inside the liver (hepatic) and after the liver (post-hepatic). Examples of pre-hepatic causes of portal hypertension include splenic vein thrombosis, portal vein thrombosis, congenital stenosis of the portal vein, extrinsic compression of the portal vein and arterio-venous fistulae. Hepatic causes other than liver cirrhosis include nodular regenerative hyperplasia, congenital hepatic fibrosis, peliosis hepatis, polycystic liver disease, idiopathic portal hypertension, toxicity from vitamin A or cyanamide, arsenic, copper sulphate or vinyl chloride polymer poisoning, granulomatous liver disease (sarcoidosis, primary biliary cirrhosis, tuberculosis, schistosomiasis), amyloidosis, mastocytosis, Rendu-Osler Weber syndrome, liver infiltration from haematological malignancies, acute fatty liver of pregnancy, severe acute viral or alcoholic hepatitis, chronic active hepatitis, hepatocellular carcinoma and veno occlusive disease of the liver. Post hepatic causes include hepatic vein thrombosis (Budd Chiari Syndrome), congenital malformations and thrombosis of the inferior vena cava, constrictive pericarditis and tricuspid valve disease. These conditions can lead to the development of oesophageal and gastric varices which can result in life threatening bleeding.

The disadvantage of current practice for portal pressure measurements is that it relies on highly invasive and costly techniques that need to be performed by highly skilled operators. For these reasons, portal pressure measurements are not done routinely, even in large hospitals specialising in liver disease.

The development of new non-invasive ways to assess portal hypertension could revolutionise the care of patients with cirrhosis because this would allow repeated measures over time to monitor how the liver disease and portal hypertension progress. In turn, this would allow timely interventions to prevent the development of complications.

Currently, there are very few non-invasive diagnostic methods for liver disease. Ultrasonography is not specific, is not sensitive in early disease, and is of limited efficacy in obese patients. Transient elastography can aid in quantifying fibrosis, but is also of limited use in large patients due to reduced acoustic windows (Varghese et al. (2002)). Magnetic resonance (MR) elastography could also be used, but it is expensive, operator-dependant and requires additional hardware (Bohte et al. (2014)). There are currently no clinical MR protocols that do not use intravenous contrast and which have been shown to diagnose parenchymal liver disease.

### SUMMARY

The invention addresses this need for a non-invasive and accurate way of assessing portal pressure or the presence of portal hypertension.

The inventors have shown that an indication of vascular pressure or changes in vascular pressure can be obtained using MRI mapping of visceral tissues. More specifically, spleen T1 relaxation time or T2* relaxation times can assist in the evaluation of portal pressure. Spleen T1 relaxation times or T2* measurements can also be used to in assist in the assessment of liver fibrosis. Additionally, liver T1 measurements (which increase with liver scarring) correlate with portal pressure measurements and thus can help in the evaluation of portal hypertension. Furthermore, liver T1 increases in the presence of vascular congestion in the liver and this could be used to assist in the assessment of central venous pressure or in the identification of blood flow obstructions in the hepatic vein and inferior vena cava.

The inventors are the first to demonstrate that an indication of the portal pressure or the presence of portal hypertension can be obtained indirectly by T1 or T2* relaxometry/mapping by MRI in a remote organ such as the liver or spleen. This discovery opens the door to a number of related inventions.

One aspect of the invention therefore relates to a novel method of assessing portal pressure using T1 or T2* relaxometry or mapping with MRI scans. T1 and T2* relaxometry and T1 and T2* mapping have not previously been used in the assessment of portal hypertension. The invention can be used for the assessment of patients with liver cirrhosis to determine whether or not they have portal hypertension. The invention can also be used to assess the portal pressure in patients with non-cirrhotic portal hypertension. The invention can also be used to assess the presence or degree of liver fibrosis.

The methods of the invention have significant advantages over the currently available methods since the MRI-based methods of the invention are quicker, completely noninvasive, do not require the patient to be admitted to hospital and also provide additional information on liver and spleen anatomy. The MRI scans could be performed on routine clinical MRI scanner without the installation of new hardware.

It is therefore an object of the invention to provide a non-invasive and accurate method of assessing portal pressure which may be useful for determining the presence or absence of portal hypertension in patients with liver cirrhosis.

It is another object of the invention to provide a non-invasive and accurate method of assessing portal pressure which may be useful for determining the presence or severity of liver fibrosis.

In a first embodiment, the invention provides a method of obtaining an indication of the vascular pressure in the blood vessels connecting a first organ to a second organ by obtaining an MRI measurement from the second organ, wherein the vascular pressure in the blood vessels between the two organs is correlated with the MRI measurement in the second organ, and wherein:

(a) the first organ is the liver and the second organ is the spleen; or
(b) the first organ is the heart and the second organ is the liver.

In one embodiment, the invention provides a method of obtaining an indication of the vascular pressure in a splanchnic vein of a subject, the method comprising the steps of:

(a) correlating an MRI measurement obtained from the subject's spleen or liver, or a value derived therefrom, with
(b) the pressure in a splanchnic vein of the subject, thereby obtaining an indication of the vascular pressure in the splanchnic vein of the subject.

In a further embodiment, the invention provides a method of obtaining an indication of the vascular pressure in a splanchnic vein of a subject, the method comprising the steps of:

(a) determining from an MRI measurement obtained from the subject's spleen or liver, or a value derived therefrom, an indication of the pressure in the splanchnic vein of the subject.

Preferably, the determining step comprises comparing the MRI measurement obtained from the subject's spleen or liver, or a value derived therefrom, with corresponding MRI measurements obtained from one or more control subjects with defined splanchnic vein pressures, thereby obtaining an indication of the vascular pressure in the splanchnic vein of the subject.

In a further embodiment, the invention provides a method of obtaining an indication of the pressure in the hepatic portal vein of a subject, the method comprising the steps of: obtaining an MRI measurement from the subject's spleen or liver, or a value derived therefrom; and correlating the measurement obtained in step (a) with the pressure in the hepatic portal vein.

In some embodiments, the correlating step preferably comprises comparing the MRI measurement obtained from the subject's spleen or liver, or the value derived therefrom, with data previously obtained from corresponding MRI measurements or values from one or more control subjects with defined hepatic portal vein pressures.

In other embodiments, the correlating step preferably comprises comparing the MRI measurement obtained from the subject's spleen or liver, or a value derived therefrom, with corresponding MRI measurements or values which have been obtained from one or more control subjects, wherein those control MRI measurements or values have been correlated with the hepatic portal vein pressures in those control subjects.

In a further embodiment, the invention provides a method of obtaining an indication of the pressure in the hepatic portal vein of a subject, the method comprising the steps of:

(a) obtaining an MRI measurement from the subject's spleen or liver, or a value derived therefrom; and
(b) determining from the MRI measurement or value in step (a) an indication of pressure in the hepatic portal vein.

In a further embodiment, the invention provides a method of obtaining an indication of hypertension in a splanchnic vein of a subject, the method comprising the steps of comparing:

(a) an MRI measurement obtained from the subject's spleen or liver, or a value derived therefrom; and
(b) the corresponding measurement or value obtained in a control without hypertension in the splanchnic vein,
wherein an increase in the measurement or value obtained in step (a) compared to the corresponding measurement or value in step (b) is indicative of hypertension in the splanchnic vein of the subject.

More preferably, the method is for obtaining an indication of hypertension in the hepatic portal vein.

In some embodiments, the finding of hypertension is measured using the Hepatic Wedge Pressure (HWP), the Free Hepatic Vein Pressure (FHVP), or the Hepatic Vein Pressure Gradient (HVPG).

In a further embodiment, the invention provides a method of obtaining an indication of a change in pressure in a splanchnic vein of a subject, the method comprising the steps of comparing:

(a) an MRI measurement obtained from the subject's spleen or liver, or a value derived therefrom; and
(b) a corresponding previously-obtained MRI measurement from the subject's spleen or liver, or a corresponding value derived therefrom,
wherein an increase in the measurement or value obtained in step (a) compared to the corresponding measurement or value in step (b) is indicative of an increase in pressure in the splanchnic vein of the subject, and
wherein a decrease in the measurement or value obtained in step (a) compared to the corresponding measurement or value in step (b) is indicative of a decrease in pressure in the splanchnic vein of the subject.

More preferably, the method is for obtaining an indication of a change in pressure in the hepatic portal vein.

In some embodiments, the pressure is the Hepatic Wedge Pressure (HWP), the Free Hepatic Vein Pressure (FHVP) or the Hepatic Vein Pressure Gradient (HVPG).

In a further embodiment, the invention provides a method of obtaining an indication of the prognosis of a subject with liver disease or portal hypertension, the method comprising the steps of comparing:

(a) an MRI measurement obtained from the subject's spleen or liver, or a value derived therefrom, with
(b) a corresponding previously-obtained MRI measurement obtained from the subject's spleen or liver, or a corresponding value derived therefrom,
wherein an increase in the measurement or value obtained in step (a) compared to the corresponding measurement or value obtained in step (b) is indicative of a decline in the prognosis for the subject, and
wherein a decrease in the measurement or value obtained in step (a) compared to the corresponding measurement or value in step (b) is indicative of an improvement in the prognosis for the subject.

In a further embodiment, the invention provides a method of obtaining an indication of the efficacy of a drug which is being used to treat liver disease or portal hypertension in a subject, the method comprising the steps of:

(a) comparing first and second MRI measurements obtained from the subject's spleen or liver, or values derived therefrom,
wherein the drug has been administered to the subject in the interval between the taking of the first and second MRI measurements,
wherein an increase in the second measurement or value compared to the first measurement or value is indicative of a lack of efficacy of the drug and
wherein a decrease in the second measurement or value compared to the first measurement or value is indicative of the efficacy of the drug.

When first and second measurements or values are being compared, the first and second measurements or values will have been taken/derived under the same conditions.

In a further embodiment, the invention provides a method of obtaining an indication of fibrosis in the liver of a subject, the method comprising the steps of correlating:

(a) an MRI measurement obtained from the subject's spleen, or from a value derived therefrom (preferably a value derived from MRI measurements from the subject's spleen and liver), with
(b) the degree of liver fibrosis, thereby obtaining an indication of fibrosis in the liver of the subject.

Preferably, the correlating step comprises correlating the MRI measurement or value obtained from the subject's spleen with corresponding MRI measurements or values obtained from one or more control subjects with defined levels of liver fibrosis.

In a further embodiment, the invention provides a method of obtaining an indication of fibrosis in the liver of a subject, the method comprising the steps of:

(a) determining from an MRI measurement obtained from the subject's spleen, or from a value derived therefrom, an indication of the degree of liver fibrosis.

Preferably, the determining step comprises correlating the MRI measurement obtained from the subject's spleen, or from a value derived therefrom (preferably a value derived from a MRI measurement of the subject's spleen and liver) with corresponding MRI measurements or values obtained from one or more control subjects with defined levels of liver fibrosis, thereby obtaining an indication of the level of liver fibrosis.

In a further embodiment, the invention provides a method of obtaining an indication of cirrhosis in the liver of a subject, the method comprising the steps of correlating:

(a) an MRI measurement obtained from the subject's spleen, or a value derived therefrom, with
(b) the severity of liver cirrhosis, thereby obtaining an indication of cirrhosis in the liver of the subject.

Preferably, the correlating step comprises correlating the MRI measurement obtained from the subject's spleen, or a value derived therefrom, with corresponding MRI measurements or values obtained from one or more control subjects with defined severity of liver cirrhosis.

In a further embodiment, the invention provides a method of obtaining an indication of cirrhosis in the liver of a subject, the method comprising the steps of:

(a) determining from an MRI measurement obtained from the subject's spleen, or a value derived therefrom, an indication of the severity of liver cirrhosis.

Preferably, the determining step comprises correlating the MRI measurement or value obtained from the subject's spleen with corresponding MRI measurements or values obtained from one or more control subjects with defined severity of liver cirrhosis, thereby obtaining an indication of the severity of liver cirrhosis.

In a further embodiment, the invention provides a method of obtaining an indication of the stage of liver disease in a subject, the method comprising the step of correlating:

(a) an MRI measurement obtained from the subject's spleen, or a value derived therefrom, with
(b) the stage of the liver disease in the subject.

Preferably, the correlating step comprises correlating the MRI measurement or value obtained from the subject's spleen with corresponding MRI measurements or values obtained from one or more control subjects with defined stages of liver disease.

In a further embodiment, the invention provides a method of obtaining an indication of the stage of liver disease in a subject, the method comprising the step of:

(a) determining from an MRI measurement obtained from the subject's spleen, or a value derived therefrom, an indication of the stage of liver disease in the subject.

Preferably, the determining step comprises correlating the MRI measurement or value obtained from the subject's spleen with corresponding MRI measurements or values obtained from one or more control subjects with defined stages of liver disease, thereby obtaining an indication of the stage of liver disease in the subject.

In some embodiments, the method obtains an indication of the stage of liver fibrosis.

In other embodiments, the method obtains an indication of the stage of liver cirrhosis.

In a further embodiment, the invention provides a method of obtaining an indication of a change in the stage of the liver disease in a subject, the method comprising the steps of comparing:

(a) an MRI measurement obtained from the subject's spleen, or a value derived therefrom; and
(b) a corresponding previously-obtained MRI measurement or value from the subject's spleen;
wherein an increase in the measurement or value obtained in step (a) compared to the corresponding measurement or value in step (b) is indicative of an increase the stage of the liver disease in the subject, and
wherein a decrease in the measurement or value obtained in step (a) compared to the corresponding measurement or value in step (b) is indicative of a decrease in the stage of the liver disease in the subject.

In some embodiments, method obtains an indication of the change in the stage of liver fibrosis.

In other embodiments, the method obtains an indication of the change in the stage of liver cirrhosis.

In a further embodiment, the invention provides a method of obtaining an indication of the central venous pressure of a subject, the method comprising the steps of correlating:

(a) an MRI measurement obtained from the subject's liver, or a value derived therefrom, with
(b) the central venous pressure, thereby obtaining an indication of the central venous pressure.

Preferably, the correlating step comprises correlating the MRI measurement obtained from the subject's liver, or value derived therefrom, with corresponding MRI measurements, or values derived therefrom, obtained from one or more control subjects with defined central venous pressure.

In a further embodiment, the invention provides a method of obtaining an indication of the central venous pressure of a subject, the method comprising the steps of:

(a) determining from an MRI measurement obtained from the subject's liver, or a value derived therefrom, an indication of the central venous pressure.

In some embodiments, the central venous pressure is the pressure in the hepatic vein, the inferior vena cava or the superior vena cava, or the right atrium.

In some preferred embodiments, the MRI measurement has been obtained from the subject's liver.

This method may also be useful to obtain an indication of pulmonary hypertension or right heart failure or congestive heart failure.

In a further embodiment, the invention provides a method of obtaining an indication of the central venous pressure of a subject, the method comprising the steps of:

(a) obtaining an MRI measurement from the subject's liver, or a value derived therefrom; and
(b) correlating the measurement or value obtained in step (a) with the central venous pressure of the subject.

In a further embodiment, the invention provides a method of obtaining an indication of the central venous pressure of a subject, the method comprising the steps of:

(a) obtaining an MRI measurement from the subject's liver or a value derived therefrom; and
(b) determining from the MRI measurement or value in step (a) an indication of central venous pressure.

In a further embodiment, the invention provides a method of obtaining an indication of increased central venous pressure in a subject, the method comprising the steps of comparing:

(a) an MRI measurement obtained from the subject's liver, or a value derived therefrom; and
(b) the corresponding measurement or value obtained from a control without increased central venous pressure,
wherein an increase in the measurement or value obtained in step (a) compared to the corresponding measurement or value obtained from the control is indicative increased central venous pressure in the subject.

In some embodiments, the increased central venous pressure in the subject is increased pressure in the hepatic vein, the inferior vena cava, the superior vena cava or the right atrium.

In a further embodiment, the invention provides a method of obtaining an indication of a change in the central venous pressure of a subject, the method comprising the steps of comparing:

(a) an MRI measurement obtained from the subject's liver, or a value derived therefrom; and
(b) a corresponding measurement or value previously-obtained from the subject's liver,
wherein an increase in the measurement or value obtained in step (a) compared to the corresponding measurement or value in step (b) is indicative of an increase in the central venous pressure of the subject, and
wherein a decrease in the measurement or value obtained in step (a) compared to the corresponding measurement or value in step (b) is indicative of a decrease in the central venous pressure of the subject.

In some embodiments, the central venous pressure is the pressure in the hepatic vein, the inferior vena cava, the superior vena cava or the right atrium.

In a further embodiment, the invention provides a method of obtaining an indication of the prognosis of a subject with heart disease, the method comprising the steps of comparing:

(a) an MRI measurement obtained from the subject's liver, or a value derived therefrom, with
(b) a corresponding previously-obtained MRI measurement or value from the subject's liver,
wherein an increase in the measurement or value obtained in step (a) compared to the corresponding measurement or value in step (b) is indicative of a decline in the prognosis for the subject, and
wherein a decrease in the measurement obtained in step (a) compared to the corresponding measurement or value in step (b) is indicative of an improvement in the prognosis for the subject.

In a further embodiment, the invention provides a method of obtaining an indication of the efficacy of a drug which is being used to treat heart disease in a subject, the method comprising the steps of:

(a) comparing first and second MRI measurements from the subject's liver, or values derived therefrom,
wherein the drug has been administered to the subject in the interval between the first and second MRI measurements,
wherein an increase in the second measurement or value compared to the first measurement or value is indicative of a lack of efficacy of the drug and
wherein a decrease in the second measurement or value compared to the first measurement or value is indicative of the efficacy of the drug.

In a further embodiment, the invention provides a system or apparatus comprising at least one processing means arranged to carry out the steps of a method of the invention.

The processing means may, for example, be one or more computing devices and at least one application executable in the one or more computing devices. The at least one application may comprise logic to carry out the steps of a method of the invention.

In a further embodiment, the invention provides a carrier bearing software comprising instructions for configuring a processor to carry out the steps of a method of the invention.

As will readily be appreciated by the skilled artisan, the methods of the invention may be computer-implemented.

As used herein, the term “vascular pressure” refers to pressure in any vasculature, wherein the vasculature comprises veins, arteries, capillary networks or any combination thereof.

In some embodiments, the methods of the invention may be used for assessing vascular pressure within the splanchnic veins, i.e. the veins from the gastrointestinal tract (superior and inferior mesenteric veins), spleen (splenic vein) and pancreas that join to form the portal vein which drains to the liver.

Preferably, the vascular pressure is the pressure in the hepatic portal vein.

In some embodiments, the vascular pressure is the Hepatic Wedge Pressure (HWP), the Free Hepatic Vein Pressure (FHVP) or the Hepatic Vein Pressure Gradient (HVPG).

In some embodiments, the methods of the invention may be used for assessing the central venous pressure. The central venous pressure reflects the amount of blood returning to the heart and the ability of the heart to pump it into the arterial circulation.

In some embodiments, the method is for obtaining an indication of increased pressure in the hepatic vein.

In some embodiments, the method is for obtaining an indication of the right atrial pressure.

The subject may be any animal, preferably a mammal, most preferably a human.

In some embodiments, the subject may be one with liver disease, preferably one with liver fibrosis or liver cirrhosis.

In some embodiments, the subject may be one with liver disease and portal hypertension, preferably one with cirrhosis or liver fibrosis.

In some embodiments, the subject may be one with portal hypertension, preferably one with non-cirrhotic portal hypertension or pre-hepatic portal hypertension or hepatic portal hypertension or post hepatic portal hypertension.

In some embodiments, the subject may be one with heart disease, preferably one with right heart failure or congestive heart failure or congenital heart disease or constrictive pericarditis or tricuspid valve disease or valvular heart disease.

In some embodiments, the subject may be one with liver disease that is a consequence of heart disease, preferably one with cardiac liver cirrhosis.

In some embodiments, the subject may be one with increased central venous pressure. The magnetic resonance imaging (MRI) measurement is one which is obtained or has been obtained from the subject's spleen or liver or from an MRI image which is obtained or has been obtained of the subject's spleen or liver.

In some embodiments, the MRI measurement may be a value that is derived from the individual liver and/or spleen MRI measurements using mathematical formulas, algorithms, databases and/or look-up tables.

In some embodiments, the MRI measurement may be a spectroscopic measurement of T1 , T2 or T2*, localised to the liver and/or spleen.

In a preferred embodiment, the value is derived from MRI measurements obtained from the liver or spleen or from images of the liver and/or the spleen.

Preferably, the value is derived from MRI measurements obtained from the subject's liver or spleen or from images of the subject's spleen and liver. More preferably, the value is derived from or based on the sum of the T1 measurements obtained from images of the subject's spleen and liver. Most preferably, the value is the sum of the T1 measurements obtained from the subject's spleen and liver or from images of the subject's spleen and liver. For example, the value may be the sum of the liver and spleen T1 measurements for the assessment of fibrosis.

In some embodiments of the invention, the method comprises the step of obtaining an MRI measurement from the subject's spleen or liver or from an image of the subject's spleen or liver.

In other embodiments of the invention, the method comprises the step of deriving a value from MRI measurements obtained from the subject's spleen or liver or from images of the subject's spleen and/or liver.

In MRI, tissue contrast is generated by a combination of intrinsic tissue properties such as spin-lattice (T1) and spin-spin (T2) relaxation times, and extrinsic properties such as imaging strategies and settings. Signal intensity in conventional MR images is displayed on an arbitrary scale, and thus is generally not adequate for direct comparisons. T1 relaxation times depend on the composition of tissues. T1 relaxation times exhibit characteristic ranges of normal values at a selected magnetic field strength. Deviation from established ranges can thus be used to quantify the effects of pathological processes.

In preferred embodiments of the invention, the MRI method used is T1 mapping or T1 relaxometry or T2 imaging or T2 mapping or 12* imaging or 12* mapping. Therefore the MRI measurement obtained may be a T1 or T2 or T2* value.

T1 relaxometry is an MRI technique that measures T1 relaxation time. T1 relaxation time is an inherent property of tissues and organs that can be measured using MRI scans. T1 relaxation time increases with increases in extracellular fluid in the organs where it is measured. Extracellular fluid can accumulate in tissues and organs for three main reasons: scarring, inflammation and increased pressure/engorgement of the tissues. When assessing a specific organ, for example the liver or spleen, the T1 relaxation time for each pixel location can be mapped onto a quantitative image, forming a T1 map, the basis of this technique. The resulting T1 could be corrected for the confounding presence of iron, something that is likely to result in more accuracy.

In the liver, T1 relaxation time increases with increasing burden of scarring. As more scarring accumulates in the liver, more pressure accumulates in the vessels of the gut, liver and spleen (portal hypertension).

As pressure accumulates in the spleen, the organ gets engorged and more extracellular fluid accumulates and this leads to an increased T1 relaxation time. If the spleen remains engorged for a long time, as is the case in patients with liver disease, then the spleen also accumulates scarring which increases extracellular fluid and consequently the T1 relaxation time even more.

The same pathological changes can be observed in the spleen independent of the underlying cause of portal hypertension. Therefore T1 mapping of the spleen could be used to assess portal pressure in all cases of portal hypertension (pre-hepatic, hepatic and post hepatic).

In the liver, T1 relaxation time increases if the organ becomes congested with blood. Blood congestion in the liver can result if there is an obstruction to the outflow of blood from the liver (e.g. obstruction in the hepatic vein or the inferior vena cava). The liver can become congested with blood from any condition that leads to increases in central venous pressure (e.g. right heart failure, congestive heart failure, congenital heart disease, constrictive pericarditis). All the causes of post hepatic portal hypertension will also cause liver congestion. On liver biopsy congestion with blood manifests as sinusoidal dilatation.

In any one or more of the embodiments of the invention, any T1 mapping method may be applied for acquiring MR relaxometry measurements or data. For example, repeated inversion recovery (IR) experiments or a modified Look Locker inversion (MOLLI) recovery pulse sequence may be used as the T1 mapping protocol. In one or more further embodiments, among others, where a shortened breath-hold is desired, the spin-lattice (T1) mapping can be performed using a shortened modified Look Locker inversion recovery (Sh-MOLLI) sequence comprising performing consecutive inversion-recovery (IR) experiments that include front-loaded sampling followed by one or more subsequent samples and conditionally including the subsequent one or more samples for the T1 mapping based on empirical relationships between the estimated spin-lattice relaxation time T1 , heart rate, heart beat period or experimentally achieved relaxation recovery times or degrees, and estimated fit error associated with the subsequent experiments and samples.

In any one or more embodiments the spin-lattice (T1) mapping can be performed using consecutive inversion-recovery (IR) experiments, wherein the consecutive IR experiments comprise a first IR experiment, a second IR experiment, and a third IR experiment, the first IR experiment comprising a number of samples exceeding a number of samples of both the second IR experiment and the third IR experiment. The method further comprises conditionally processing the samples in the first, second, and third IR experiments.

These examples are not exhaustive. T1 mapping could also be carried out using, for example, saturation recovery, multiple-flip-angle, or MR fingerprinting methods.

In some embodiments of the invention, the method may further comprise the step of correction of the MRI measurement or value for iron overload from a measurement of iron content obtained from the liver or spleen tissue of the subject.

In preferred embodiments, the tissue may be measured for iron content using one or more of T2 mapping, T2* mapping, measuring one or more blood biomarkers such as ferritin, transferrin, transferrin saturation, hepcidin, soluble transferring receptor (sTfR) index (sTfR/log ferritin), or MR spectroscopy. For example, the width of the 1 H MRS spectra can indicate higher than normal iron loads. In some embodiments, excess iron may be determined from stainable iron visible in a liver tissue biopsy, such as a positive results on a Perl's stain, or by measuring dry weigh iron from a separate liver biopsy (normal liver has less than 3 mmols iron per 00 g of liver tissue).

Where excess iron or iron deficit is found the measured MRI value can be corrected by applying a correction factor to the measured MRI values. Where the iron concentration is normal, it may not be necessary to apply a correction factor, in which case the measured MRI value can serve as a biomarker for vascular pressure without correction.

For example, T1 measurements in an organ increase if extracellular fluid increases in that organ, as is the case in inflammation, fibrosis and vascular congestion. However, iron deposited in the organ competes with this effect, leading to a reduction in the measured T1. A correction algorithm can be applied to remove the bias introduced by the presence of iron and this yields the iron corrected T1 metric (cT1; the T1 that would have been measured if the iron concentration was normal).

In some aspects of the invention, the measurement, value or indication obtained of the vascular pressure provides information regarding the state of an associated tissue or organ or an indicator of the disease state of an associated tissue or organ. The associated organ may be distal to the vascular tissue where the vascular pressure is assessed.

In preferred embodiments, the vascular pressure being assessed is the portal pressure and the associated organ or tissue is the liver. In such an embodiment, the portal pressure provides an indication of the status or health of the liver, such as the presence and/or degree of liver fibrosis or liver cirrhosis.

Methods of the invention may also be used for assessing vascular pressure associated with visceral tissues other than the liver. Therefore, a further aspect of the invention comprises a method of obtaining an indication of vascular pressure associated with a first visceral tissue by imaging a second visceral tissue.

The methods of the invention may also be used to assess central venous pressure or pressure in vasculature in close proximity to the heart such as the vena cava by imaging the liver using MRI methods. Use of these methods of the invention would provide an indication useful for the assessment of the presence or degree of increased central venous pressure or right-sided heart failure or congestive heart failure.

In one embodiment, the invention provides a method of obtaining an indication of the vascular pressure in the splanchnic vein of a subject, the method comprising the steps of:

(a) correlating an MRI measurement obtained from the subject's spleen or liver, or a value derived therefrom, with
(b) the pressure in a splanchnic vein of the subject, thereby obtaining an indication of the vascular pressure in the splanchnic vein of the subject.

The indication of the vascular pressure in the splanchnic vein of the subject will, in general, be based on the MRI measurement obtained from the subject's spleen or liver, or a value derived therefrom.

In some embodiments, the correlating step preferably comprises comparing the MRI measurement obtained from the subject's spleen or liver, or a value derived therefrom, with data previously-obtained from corresponding MRI measurements or values from MRI images from one or more control subjects with defined splanchnic vein pressures. In other embodiments, the correlating step preferably comprises comparing the MRI measurement obtained from the subject's spleen or liver, or a value derived therefrom, with corresponding MRI measurements or values which have been obtained from one or more control subjects, wherein those control MRI measurements or values have been correlated with the splanchnic vein pressures in those control subjects.

The MRI measurement or value from one or more control subjects may be been correlated, for example, with the Hepatic Wedge Pressure (HWP), Free Hepatic Vein Pressure (FHVP), the Hepatic Vein Pressure Gradient (HVPG) or the Right Atrial Pressure.

The data previously-obtained from one or more control subjects may be in any suitable form, e.g. graphs or look-up tables or databases or it may be in the form of a mathematical equation which correlates the MRI measurement or value with the pressure or gradient or disease severity. The data may be in a computer-encoded or computer-readable format. The data may be in the form of mathematical equations, for example equations derived from the individual MRI measurements of liver T1 , T2 and T2* and spleen T1 , T2 and T2* values.

For example, MRI value might be the sum of liver cT1 and spleen T1.

The value may also incorporate other measurements, such as spleen length, spleen volume, maximum spleen cross sectional diameter or blood test results (e.g. platelet count).

As used herein, the term “control” relates to an individual or group of individuals of the same species as the subject being tested. For example, if the subject is a human, the control will also be a human.

The control subjects have defined splanchnic venous pressures and central venous pressures which have been measured using methods other than MRI, e.g. using an invasive technique such as the use of a vascular catheter and pressure transducer.

In a further embodiment, the invention provides a method of obtaining an indication of the vascular pressure in the splanchnic vein of a subject, the method comprising the step of:

(a) determining from an MRI measurement obtained from the subject's spleen or liver, or a value derived therefrom, an indication of the pressure in the splanchnic vein of the subject.

Preferably, the determining step comprises comparing the MRI measurement or value obtained from the subject's spleen or liver with corresponding MRI measurements or values obtained from one or more control subjects with defined splanchnic vein pressures, thereby obtaining an indication of the vascular pressure in the splanchnic vein of the subject.

As used herein, the term “corresponding MRI measurement” or “corresponding value” refers to a MRI measurement made under the same conditions as the one to which it is being compared or a value which is derived in the same manner. For example, the “corresponding MRI measurement” may refer to a MRI measurement which is made on the same organ or the same part of the organ as the one to which it is being compared.

Normal portal pressure values are generally less than about 5 mm Hg. A measurement of a Hepatic Vein Pressure Gradient (HVPG) of greater than 10 mm Hg denotes clinically-significant portal hypertension. The following portal pressure values may also be used to classify degrees of portal hypertension:

HVPG<5 mmHg: No portal hypertension,
HVPG 6-9 mmHg: Pre-clinical portal hypertension,
HVPG3≥10 mmHg: Clinically significant portal hypertension,
HVPGA\hat{ }12 mmHg: Severe portal hypertension.

An indication of portal hypertension may be an indication of a pre-hepatic condition, a hepatic condition or a post-hepatic condition.

In a further embodiment, the invention provides a method of obtaining an indication of the efficacy of a drug which is being used to treat liver disease or portal hypertension in a subject, the method comprising the steps of:

(a) comparing first and second MRI measurements, or values derived therefrom, of the subject's spleen or liver,
wherein the drug has been administered to the subject in the interval between the taking of the first and second MRI measurements,
wherein an increase in the second measurement or value compared to the first measurement or value is indicative of a lack of efficacy of the drug and
wherein a decrease in the second measurement or value compared to the first measurement or value is indicative of the efficacy of the drug.

In some embodiments of the invention, step (a) comprises administering the drug to the subject in the interval between the first and second MRI measurements.

The MRI measurements (e.g. the T1 , T2 and/or T2* measurements) are taken in a Region Of Interest (ROI) which may be automatically segmented or chosen by the operator.

For the liver, one or more of the following 4 considerations are generally made:

(a) In the acquisition of T1 maps, using one implementation of the ShMOLLI method, there is a quality assurance step where each acquisition generates an R² map for the fit of signal intensity to the exponential recovery curve (Ferreira et a/.(2012)). A region of interest is only considered for interpretation if the R² is A 99%.
(b) The ROI is placed approximately halfway between the porta hepatis and the liver surface in order to avoid interference from the fluid filled structures in the porta hepatis and subcutaneous tissue or air close to the liver surface,
(c) The ROI is placed so as to avoid visible bile ducts and blood vessels,
(d) The ROI is placed in an area that corresponds to good quality images in the T2* map in order to allow T1 and T2* quantification in the same ROI.

If a spectroscopic method is used to measure T1 , T2 or T2* then the ROI can be the average of the voxel where the measurements are taken.

In some embodiments of the invention which relate to MRI measurements in the subject's spleen, the invention excludes methods wherein the spleen has been subjected to mechanical excitation such that an oscillating stress is present in the spleen at the time that the MRI measurement is taken.

### BRIEF DESCRIPTION OF THE FIGURES

FIG. 1 shows the steps for choosing a liver Region of Interest (ROI) in transverse T1 and T2* maps of the abdomen. The region of interest was placed on the same location in the T1, R² (quality assurance component of T1 mapping) and T2* maps as illustrated here. The number of pixels, area, minimum, maximum, mean and standard deviation for each region of interest (ROI) are automatically read by the scanner console software. The mean T1 and T2* value for each ROI was used in the final analysis.

FIG. 2 shows the associations between spleen T1 and (a) Hepatic Venous Pressure Gradient (HVPG) and (b) Hepatic Wedge Pressure (HWP).

FIG. 3 shows that spleen T2* could identify patients with clinically significant portal hypertension (HVPG\hat{ }IOmmHg). The median spleen T2* for patients with HVPG\hat{ }IOmmHg was 37.2 ms compared to 22.9 ms for patients with a HVPG<IOmmHg (p=0.036). HVPG: Hepatic Vein Pressure Gradient

FIG. 4 shows associations between Liver T1 and (a) Hepatic Venous Pressure Gradient (HVPG) and (b) Hepatic Wedge Pressure (HWP).

FIG. 5 shows that spleen T1 measurements in patients with a normal spleen iron (defined as a spleen T2*>19 ms) show a highly significant association with histological assessment of liver fibrosis.

FIG. 6 shows that spleen T1 values for patients with low spleen iron (T2*>19 ms). Mean±SD for spleen T1 for each group is as follows: Ishak 0-2: 1278 ms±103; Ishak 3-4: 1325 ms ±82 and Ishak 5-6: 1439 ms±72. One way analysis of variance with Bonferroni's correction showed significant differences between Ishak 5-6 and Ishak 0-2 and Ishak 3-4 (Table 1).

FIG. 7 shows that the sum of spleen T1 and liver corrected T1 measurements in patients with a normal spleen iron (defined as a spleen T2*>19 ms) show a highly significant association with histological assessment of liver fibrosis.

FIG. 8 shows liver cT1 and sinusoidal dilatation in patients with Hepatitis C. The images in the top row show transverse (a) T1 and (b) T2* maps in a patient with Hepatis C and mild fibrosis (Ishak 1). The images in the bottom row show transverse (c) T1 and (d) T2* maps from a patient with Hepatitis C, mild fibrosis (Ishak 1) and sinusoidal dilatation which is a finding indicative of vascular congestion in the liver. The values measured in a region of interest on the right lobe are shown for each image and the corrected T1 (cT1) is shown on the far right for each patient. This demonstrates that the presence of sinusoidal dilatation when other factors are equal (e.g. degree of fibrosis and underlying liver pathology-hepatitis C in this case) leads to increases in the liver corrected T1 values.

FIG. 9 shows liver cT1 and sinusoidal dilatation in patients with Primary Sclerosing Cholangitis (PSC). The images in the top row show transverse (a) T1 and (b) T2* maps in a patient with PSC and mild fibrosis (Ishak 1). The images in the bottom row show transverse (c) T1 and (d) T2* maps from a patient with PSC, mild fibrosis (Ishak 1) and sinusoidal dilatation. The values measured in a region of interest on the right lobe are shown for each image and the corrected T1 (cT1) is shown on the far right for each patient. Again, this demonstrates that the presence of sinusoidal dilatation when other factors are equal (e.g. degree of fibrosis and underlying liver pathology—PSC in this case) leads to increases in the liver corrected T1 values.

### DETAILED DESCRIPTION

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of each reference set forth herein is incorporated herein by reference in its entirety.

### EXAMPLES

### Example 1

### Liver and Spleen T1 and T2* Mapping Using MRI Can Help in the Evaluation of Portal Pressure

### Background

The accumulation of liver fibrosis leads to increased hepatic vascular resistance and portal hypertension. Once portal hypertension is established in the context of liver disease, the patient is at risk of complication including, variceal bleeding, ascites, encephalopathy and renal dysfunction. Furthermore the degree of portal hypertension has important prognostic implications in many clinical situations including alcoholic hepatitis, (Rincon et al. (2007)), liver cirrhosis, (Ripoll et al. (2005)) and recent variceal bleeding (Patch et al. (1999)).

Portal pressure is usually assessed by measuring the Hepatic Vein Pressure Gradient (HVPG), which is invasive, costly and requires high expertise. Non invasive ways to assess portal hypertension have the potential to revolutionise the care of patients with liver disease as they would allow widespread use and repeated measurements to assess the progression/regression of portal hypertension over time.

A novel MRI technique has been developed for imaging the liver and spleen based on the following hypothesis:

1 . The spleen parenchyma undergoes changes which may be assessed using MRI (T1 and T2* mapping) and which may indicate the severity of portal hypertension.
2. The severity of liver fibrosis as measured by liver T1 mapping would reflect the severity of portal hypertension.

### Methods

### Patient Population and Study Design

A prospective study was conducted to assess the MR method in the evaluation of patients with portal hypertension due to chronic liver disease. The designated reference standard was the Hepatic Vein Pressure Gradient. Patients with suspected liver cirrhosis who were due to have a liver biopsy as part of their clinical care were invited to have their biopsy through the trans-jugular route with portal pressure assessment at the time of the biopsy. Patients with portal vein thrombosis and with contra-indications to MR scanning were excluded. Eleven patients were recruited in the study period (July 2013-March 2014). The patients attended for an MRI scan prior to their biopsy.

### MRI Assessment

All MR scans were performed in Oxford, UK, with the patient lying supine in a 3 Tesla system (Tim Trio, Siemens Healthcare, Germany). Patients attended for their scans having fasted for at least 4 hours. Transverse T1 and T2* maps of the abdomen were acquired and T1 and T2* measurements were documented for the liver and spleen.

### Transverse Abdominal T1 Map

A T1 relaxation time map was acquired using the Shortened Modified Look Locker Inversion recovery (shMOLLI) sequence (Piechnik et al, J Cardiovasc Magn Reson) in a transverse plane through the liver and the spleen. A subject-dependent frequency adjustment was carried out during end-expiration. The ShMOLLI sequence samples the T1 recovery curve using single-shot steady state free precession (SSFP) acquisitions using the following parameters:

TR 2.14 ms, TE 1.07 ms, flip angle of 35°, field-of-view optimised per patient, acquisition matrix 192×134-160, depending on patient, with GRAPPA acceleration of 2 with 24 reference lines, yielding a typical interpolated voxel size 0.9×0.9×8mm. Images were acquired 200 ms after the ECG R-wave and the total time for each SSFP acquisition between 169 and 197 ms, depending on the number of phase encoding steps. The variable acquisition parameters fall in the ranges used in myocardial T1 mapping at 1.5T, with associated inter-individual coefficient of variation for normal myocardial T1 of 2.2%,(Piechnik et al, J Cardiovasc Magn Reson) well within the inter-subject coefficient of variation measured in normal volunteers here of 7%.

### Transverse Abdominal T2* Mapping

A multi-gradient-echo acquisition with RF spoiling is used to calculate a T2* map of the liver. The same field-of-view as in the T1 mapping sequence is used, with a matrix size of 192×128-160, depending on patient, slice thickness of 3 mm and 2× GRAPPA acceleration, with the same 200 ms delay after the R-wave before acquisition. The image is acquired in nine segments with a TR of 26.5 ms and flip angle of 20°. Echo times are selected as far as possible such that the signals from fat and water are in phase (TE=2.46, 7.38, 12.30, 17.22 and 22.14 ms). Fat-saturation and a double-inversion-recovery black blood preparation are used.

### Region of Interest Placement—Liver

A single Region of Interest (ROI) was selected for each patient. There were 4 considerations in the choice of the ROI:

(a) As each acquisition generates an R² map for the fit of signal intensity to the exponential recovery curve the ROI was chosen in an area where R² was >99% (which was the case in all patients),
(b) The ROI was placed approximately halfway between the porta hepatis and the liver surface in order to avoid interference from the fluid filled structures in the porta hepatis and subcutaneous tissue or air close to the liver surface,
(c) The ROI was placed so as to avoid visible bile ducts and blood vessels,
(d) The ROI was placed in an area that corresponded to good quality images in the T2* map in order to allow T1 and T2* quantification in the same ROI.

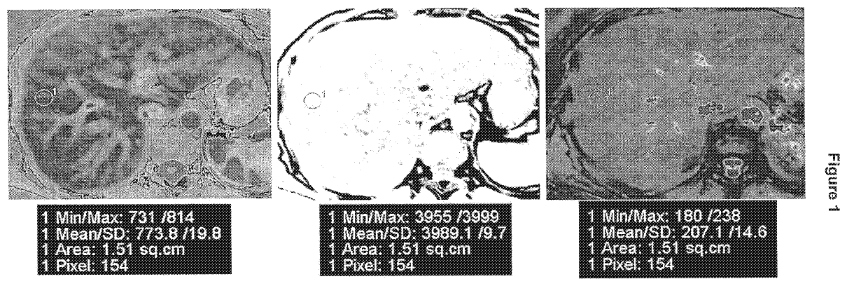

FIG. 1 illustrates the process of choosing an ROI for the liver.

### Region of Interest Placement—Spleen

The choice of the ROI for the spleen followed the same principles as described above for the liver. The ROI placement in the spleen was less complicated as the spleen is smaller, and more homogeneous than the liver. It also has no blood vessels running through its parenchyma and the step of avoiding blood vessels and bile ducts as described above for the liver (step (d) above) did not apply to the spleen.

### MR Image Analysis

Data were analysed by physicians blinded to the clinical information, using software tools available on the scanner console. For each patient one ROI was selected for the liver and one ROI for the spleen and mean T1 and T2* were recorded for each.

### Assessment of Portal Pressures

The assessment of the portal pressures was performed by a consultant interventional radiologist specialising in hepatobiliary procedures. The procedures were carried out with the patient in the supine position, under conscious sedation, after an overnight fast. After injection of local anaesthetic in the skin, a vascular catheter was inserted through an introducer into the right internal jugular vein under ultrasound guidance. This catheter was advanced into the right atrium and then the hepatic vein to measure the right atrial pressure and free hepatic vein pressure (FHVP) respectively. The catheter was then further advanced into the wedged position where the Hepatic Wedge Pressure (HWP) was taken. The Hepatic Vein Pressure Gradient was calculated as the difference between the FHVP and the HWP.

The Hepatic Vein Pressure Gradient was used to classify portal hypertension according to this schema: HVPG<5 mmHg: No portal hypertension, HVPG 6-9 mmHg: Pre-clinicai portal hypertension, HVPG>IOmmHg: Clinically significant portal hypertension, HVPG>12 mmHg: severe portal hypertension

### Statistics

Pearson's correlation and linear regression analysis was used to check for associations between variables. Summary data for each of the HVPG categories were calculated and the Mann Whitney test was used to test for differences between the groups.

### Results

### Spleen T1

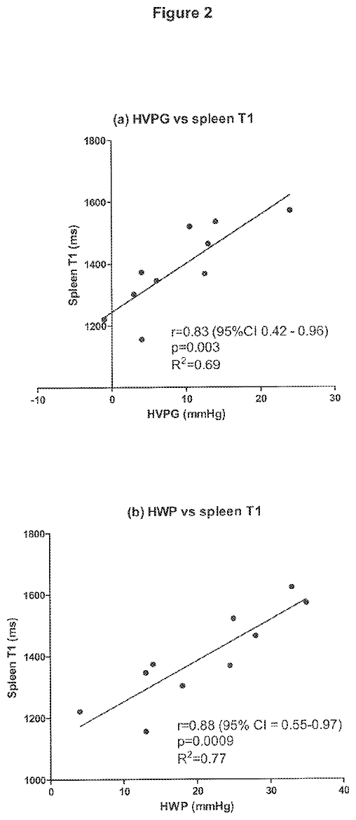

We found a highly significant association both between the HVPG (r=0.83; p=0.003, R²=0.69) and the HWP (r=0.88; p=0.0009; R²=0.77) and the spleen T1 relaxation time (FIG. 2).

### Spleen T2*

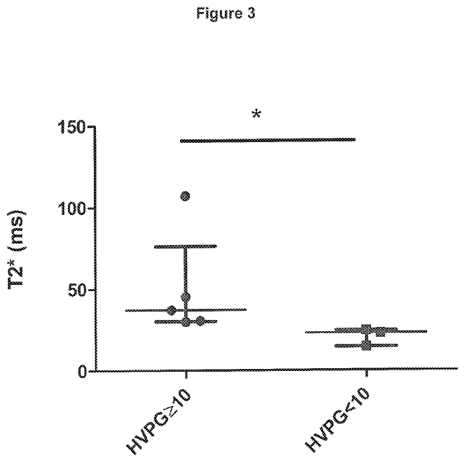

Significant differences in the spleen T2* were found between those patients with a HVPG>“I OmmHg (median 37.2 ms; IQR 30.2-76.2) and those with a HVPG <10mmHg (median 22.9 ms; IQR 14.6-24.5; p=0.036; FIG. 3).

### Liver T1

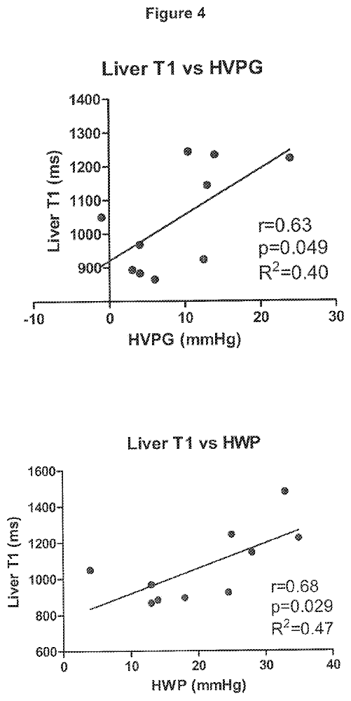

We found a statistically significant association both between the HVPG (r=0.63; p=0.049, R²=0.40) and the HWP (r=0.68; p=0.029; R²=0.47) and the liver T1 relaxation time (FIG. 4).

### Example 2

### Spleen T1 and T2* Mapping Can be Used to Help in the Assessment of Liver Fibrosis.

### Background

Changes in the severity of liver disease can result in structural and other changes in the spleen. This is particularly true for the late stages of liver disease, when portal hypertension is established. The spleen becomes engorged with portal venous blood and can be enlarged. This can be picked up on clinical examination or by using routine uitrasound scans. However, it is not known if the spleen undergoes any changes in the early stages of liver fibrosis. We hypothesised that our MR techniques for measuring spleen T1 and T2* are very sensitive and may be able to detect early changes in the spleen that occur before portal hypertension is established.

### Methods

### Study Design and Population

A prospective study of a new diagnostic MR method to evaluate the severity of liver fibrosis was conducted. The designated reference standard was histological assessment of liver fibrosis. From March 2011 to March 2014, patients referred for liver biopsy in Oxford were invited to take part in the study. Patients with contraindications for MR scanning and patients that were found to have an increased amount of iron in their spleen were excluded. A patient was considered to have an increased spleen iron if the measured T2* value was less than 19 ms. Patients attended for an MRI scan prior to their liver biopsy.

The MRI procedure for the acquisition and recording of liver T1 and T2* and spleen T1 and T2* is the same as described in example 1. In addition, an algorithm was used to correct the liver T1 values for the amount of iron present to produce the corrected T1 metric (cT1 ; Banerjee et al, J Hepatol).

### Histological Interpretation of Liver Biopsies.

The Ishak score (Ishak et al, J Hepatol) was used for the histological assessment of liver fibrosis. In this score fibrosis is scored on a scale from 0 (no fibrosis) to 6 (severe fibrosis; cirrhosis). In clinical practice patients can be subdivided into 3 groups according to their Ishak score. (Ishak 0-2: no or mild fibrosis; Ishak 3-4: moderate fibrosis; Ishak 5-6 advanced fibrosis).

### Statistics

Spearman's correlation coefficient was used to test for associations between the Ishak stages and spleen T1 and the sum of spleen T1 and liver cT1. One way analysis of variance (ANOVA) with Bonferroni's correction was used to test for differences between the spleen T1 and spleen T2* of those with Ishak 0-2, Ishak 3-4 and Ishak 5-6.

### Results

### Spleen T1

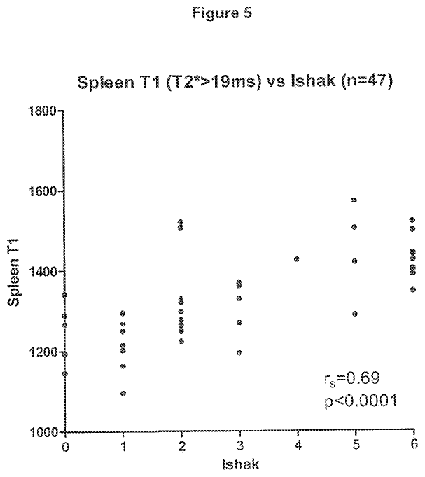

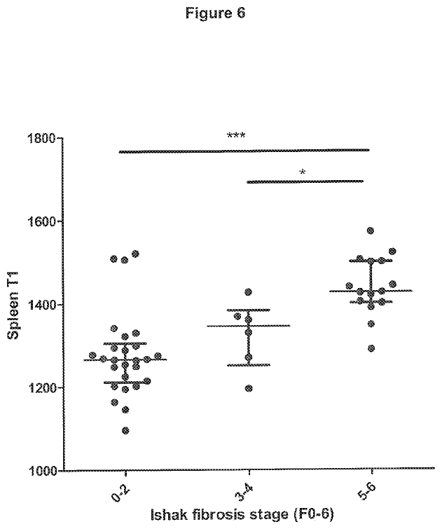

In patients with a low amount of iron in their spleen (spleen T2*>19 ms), there was a highly significant association between the spleen T1 and the degree of liver fibrosis assessed by histology (rₛ=0.69; p<0.000; FIG. 5). The mean spleen T1 values of patients with severe (Ishak 5-6), moderate (Ishak 3-4) and no or mild fibrosis (Ishak 0-2) were 1439 ms, 1352 ms and 1278 ms respectively. One way ANOVA with Bonferroni's correction showed significant differences between Ishak 5-6 and Ishak 0-2 and Ishak 3-4 (FIG. 6 and Table 1).

TABLE 1
One way analysis of variance with Bonferroni's correction for
comparison between disease severity groups for spleen T1

| Bonferroni's Multiple | Mean | | Significant? | |
|---|---|---|---|---|
| Comparison Test | Diff. | t | P < 0.05? | 95% CI of diff |
| 0-2 vs 3-4 | −47.26 | 1.135 | No | −150.9 to 56.36 |
| 0-2 vs 5-6 | −161.0 | 5.401 | Yes | −235.2 to −86.80 |
| 3-4 vs 5-6 | −113.7 | 2.561 | Yes | −224.3 to −3.202 |

TABLE 2
One way analysis of variance with Bonferroni's correction for
comparison between disease severity groups for spleen T2*

| Bonferroni's Multiple | Mean | | Significant? | |
|---|---|---|---|---|
| Comparison Test | Diff. | t | P < 0.05? | 95% CI of diff |
| 0-2 vs 3-4 | −1.219 | 0.2465 | No | −13.36 to 10.92 |
| 0-2 vs 5-6 | −19.17 | 4.071 | Yes | −30.72 to −7.614 |
| 3-4 vs 5-6 | −17.95 | 3.060 | Yes | −32.34 to −3.557 |

### Sum Liver cT1 and Spleen T1

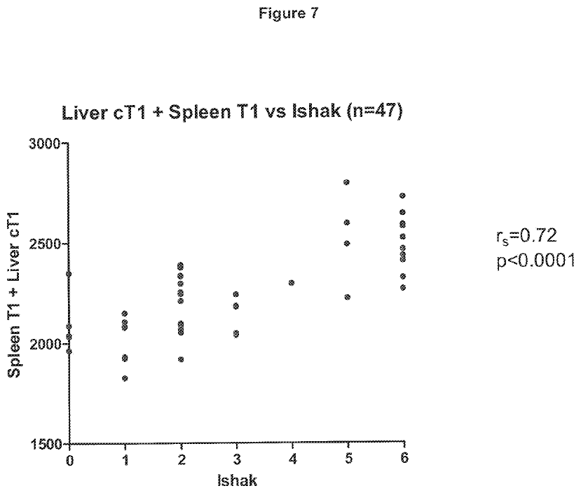

There was a highly significant correlation between the sum of spleen T1 and liver cT1 with the Ishak fibrosis score (rₛ=0.72; p<0.0001, FIG. 7). The correlation of the sum was stronger than the correlation of spleen T1 and Ishak score (rₛ=0.69; p<0.0001; FIG. 5) or between liver cT1 and Ishak (rₛ=0.61 , p<0.0001 ; data not shown).

### Example 3

### Liver T1 Measurements Can Help Evaluate Vascular Congestion in the Liver

### Background

Liver vascular congestion can result from liver venous outflow tract obstruction or from any cause of increased central venous pressure (e.g. right heart failure, congestive heart failure, constrictive pericarditis, obstruction in the intra-thoracic inferior vena cava). Vascular congestion in the liver manifest as liver sinusoidal dilatation which is detected on liver biopsies. We hypothesised that liver T1 and corrected T1 would be higher in patients who have sinusoidal dilatation on liver biopsies, compared to patients without.

### Methods

Pairs of patients with the same Ishak stage and the same underlying liver disease aetiology were identified from the cohort of patients in the studies of examples 1 and 2. The liver T1 and corrected T1 values of the patient pairs were compared to establish if the presence of sinusoidal dilatation had any impact on the observed measurements.

### Results

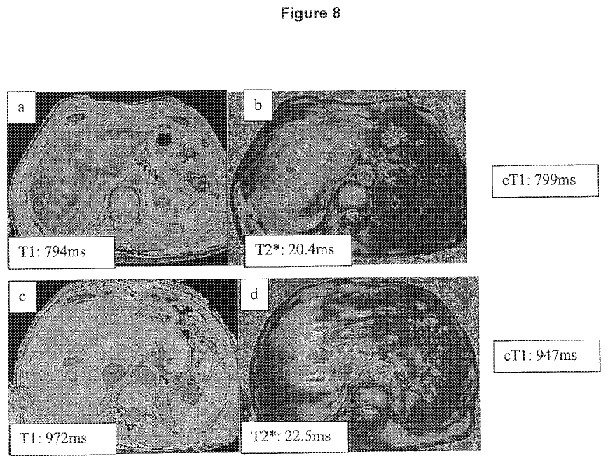

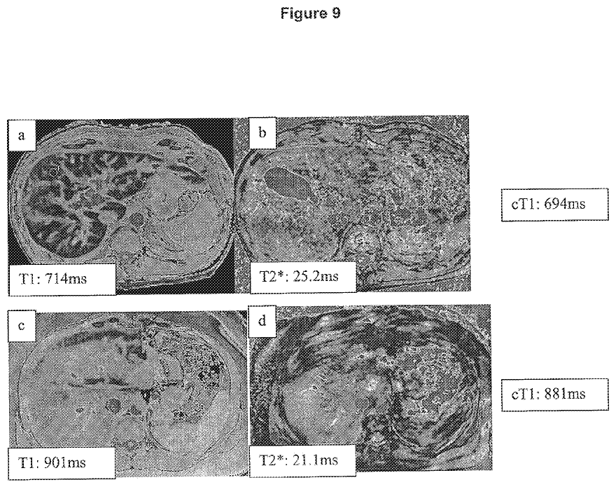

We found that the presence of sinusoidal dilatation leads to an increase in the liver T1 and corrected T1. FIG. 8 illustrates a comparison between two patients with Hepatitis C and mild fibrosis (Ishak 1), where one doesn't have sinusoidal dilatation (top row, panels a and b) the other does (bottom row, panels d and c). FIG. 9 illustrates the same situation in two patients with Primary Sclerosing Cholangitis. In both scenarios, the patient where sinusoidal dilatation is present has higher liver T1 and cT1 measures.

### Example 4

### Example of How to Assess Central Venous Pressure Using Imaging of the Liver

### Background

The liver is directly connected to the heart via the hepatic veins and the inferior vena cava. Any pathological process that leads to an increased central venous pressure can result in increased liver vascular congestion. Increases in central venous pressure can be seen in right heart failure, congestive heart failure, constrictive pericarditis and congenital heart disease.

Other causes of liver vascular congestion include liver veno-occlusive disease and venous outflow tract obstruction. Over the long term, liver vascular congestion from any cause can lead to the accumulation of fibrosis and the development of cirrhosis and liver failure.

A way of assessing liver congestion non-invasively would allow clinicians to monitor both the progression of the underlying condition causing liver congestion (e.g. right heart failure), but also the direct effect on the liver.

Liver T1 and T2* mapping and the corrected T1 metric may be used to assess liver vascular congestion and studies are designed to test this in the context of right heart failure.

### Methods

Patients who are having routine clinical investigations for the evaluation of central venous pressure and right heart function are recruited. Patients have a liver MRI for T1 and T2* mapping and measurement of liver cT1 in addition to their clinically indicated investigations which may include echocardiography, cardiac MRI and cardiac catheter studies.

Association between parameters of central venous pressure and right heart function measured on echocardiography, cardiac MRI and cardiac catheter studies and Liver T1 and cT1 are tested.

The echocardiographic parameters include:

1. Right ventricular diameter
2. Pulmonary artery pressure
3. Presence and degree of tricuspid regurgitation
The MRI parameters of include:
1. Right ventricular Ejection Fraction
2. Right ventricular end systolic and end diastolic volume
3. Right ventricular end systolic and end diastolic diameter
The cardiac catheter parameters include
1. Right atrial pressure
2. Pulmonary artery pressure
3. Pulmonary wedge pressure
4. Right ventricle end systolic and end diastolic pressure
5. Right ventricle end systolic and end diastolic volume
6. The difference between the LV end diastolic pressure and the hepatic wedge pressure.

### REFERENCES

Banerjee, R., M. Paviides, et al. “Multiparametric Magnetic Resonance for the noninvasive diagnosis of liver disease.” J. Hepatol. 2014 January; 60(1): 69-77.

Bohte, A. E., A. de Niet, et al. “Non-invasive evaluation of liver fibrosis: a comparison of ultrasound-based transient elastography and MR elastography in patients with viral hepatitis B and C.” Eur Radiol 24(3): 638-48.

Bosch, J., J. G. Abraldes, et al. (2009). “The clinical use of HVPG measurements in chronic liver disease.” Nat Rev Gastroenterol Hepatol 6(10): 573-582.

Bravo, A. A., S. G. Sheth, et al. (2001). “Liver Biopsy.” New England Journal of Medicine 344(7): 495-500.

Escorsell, A., C. Bru, et al. (1999). “Wedged hepatic venous pressure adequately reflects portal pressure in hepatitis C virus-related cirrhosis.” Hepatology 30(6): 1393-1397.

Ferreira V M, Piechnik S K, Dall'Armellina E, Karamitsos T D, Francis J M, Choudhury R P et al Non-contrast T1 -mapping detects acute myocardial edema with high diagnostic accuracy: a comparison to T2-weighted cardiovascular magnetic resonance J

Cardiovasc Magn Reson 2012; 14:42.

Ishak, K., A. Baptista, et al. (1995). “Histological grading and staging of chronic hepatitis.” J Hepatol 22(6): 696-9.

Merkel, C. and S. Montagnese “Hepatic venous pressure gradient measurement in clinical hepatology.” Digestive and Liver Disease 43(10): 762-767.

Patch, D., A. Armonis, et al. (1999). “Single portal pressure measurement predicts survival in cirrhotic patients with recent bleeding.” Gut 44(2): 264-9.

Piechnik, S., V. Ferreira, et al. (2010). “Shortened Modified Look-Locker Inversion recovery (ShMOLLI) for clinical myocardial T1 -mapping at 1.5 and 3 T within a 9 heartbeat breathhold.” Journal of Cardiovascular Magnetic Resonance 12(1): 69.

Piechnik, S. K., V. M. Ferreira, et al. “Normal variation of magnetic resonance T1 relaxation times in the human population at 1.5 T using ShMOLLI.” J Cardiovasc Magn Reson 15: 13.

Rincon, D., O. Lo Iacono, et al. (2007). “Prognostic value of hepatic venous pressure gradient for in-hospital mortality of patients with severe acute alcoholic hepatitis.” Aliment Pharmacol Ther 25(7): 841-8.

Ripoll, C, R. Banares, et al. (2005). “Influence of hepatic venous pressure gradient on the prediction of survival of patients with cirrhosis in the MELD Era.” Hepatology 42(4): 793-801.

Varghese, T., J. A. Zagzebski, et al. “Elastographic imaging using a handheld compressor.” Ultrason Imaging. 2002 January; 24(1):25-35.

Therefore, the following is claimed:

1. A method of obtaining an indication of the vascular pressure in the blood vessels connecting a first organ to a second organ by obtaining an MRI measurement from the second organ, wherein the vascular pressure in the blood vessels between the two organs is correlated with the MRI measurement in the second organ, wherein:
(a) the first organ is the liver and the second organ is the spleen; or
(b) the first organ is the heart and the second organ is the liver,
and wherein the MRI measurement is T1 mapping or T1 relaxometry or T1 values (ms) or T2* imaging or T2* mapping or T2* values (ms) or T2 mapping or T2 relaxometry or T2 values (ms).

2. A method of obtaining an indication of the vascular pressure in a splanchnic vein of a subject, the method comprising the steps of:
(a) correlating an MRI measurement obtained from the subject's spleen or liver, or a value derived therefrom, with
(b) the pressure in a splanchnic vein of the subject,
wherein the MRI measurement is T1 mapping or T1 relaxometry or T1 values (ms) or T2* imaging or T2* mapping or T2* values (ms) or T2 mapping or T2 relaxometry or T2 values (ms), thereby obtaining an indication of the vascular pressure in the splanchnic vein of the subject.

3. A method of obtaining an indication of the vascular pressure in a splanchnic vein of a subject, the method comprising the steps of:
(a) determining from an MRI measurement obtained from the subject's spleen or liver, or a value derived therefrom, an indication of the pressure in the splanchnic vein of the subject, wherein the MRI measurement is T1 mapping or T1 relaxometry or T1 values (ms) or T2* imaging or T2* mapping or T2* values (ms) or T2 mapping or T2 relaxometry or T2 values (ms).

4. A method as claimed in claim 3, wherein the determining step comprises comparing the MRI measurement obtained from the subject's spleen or liver, or a value derived therefrom, with corresponding MRI measurements obtained from one or more control subjects with defined splanchnic vein pressures, thereby obtaining an indication of the vascular pressure in the splanchnic vein of the subject.

5. A method of obtaining an indication of the pressure in the hepatic portal vein of a subject, the method comprising the steps of:
(a) obtaining an MRI measurement from the subject's spleen or liver, or a value derived therefrom; and
(b) correlating the measurement obtained in step (a) with the pressure in the hepatic portal vein,
wherein the MRI measurement is T1 mapping or T1 relaxometry or T1 values (ms) or T2* imaging or T2* mapping or 12* values (ms) or T2 mapping or T2 relaxometry or T2 values (ms).

6. A method as claimed in claim 5, wherein the correlating step comprises comparing the MRI measurement obtained from the subject's spleen or liver, or the value derived therefrom, with data previously obtained from corresponding MRI measurements or values from one or more control subjects with defined hepatic portal vein pressures.

7. A method as claimed in claim 5, wherein the correlating step comprises comparing the MRI measurement obtained from the subject's spleen or liver, or a value derived therefrom, with corresponding MRI measurements or values which have been obtained from one or more control subjects, wherein those control MRI measurements or values have been correlated with the hepatic portal vein pressures in those control subjects.

8. A method of obtaining an indication of the pressure in the hepatic portal vein of a subject, the method comprising the steps of:
(a) obtaining an MRI measurement from the subject's spleen or liver, or a value derived from therefrom; and
(b) determining from the MRI measurement or value in step (a) an indication of pressure in the hepatic portal vein,
wherein the MRI measurement is T1 mapping or T1 relaxometry or T1 values (ms) or T2* imaging or T2* mapping or T2* values (ms) or T2 mapping or T2 relaxometry or T2 values (ms).

9. A method of obtaining an indication of hypertension in a splanchnic vein of a subject, the method comprising the steps of comparing:
(a) an MRI measurement obtained from the subject's spleen or liver, or a value derived therefrom; and
(b) the corresponding measurement or value obtained in a control without hypertension in the splanchnic vein,
wherein the MRI measurement is T1 mapping or T1 relaxometry or T1 values (ms) or T2* imaging or T2* mapping or T2* values (ms) or T2 mapping or T2 relaxometry or T2 values (ms),
wherein an increase in the measurement or value obtained in step (a) compared to the corresponding measurement or value in step (b) is indicative of hypertension in the splanchnic vein of the subject.

10. A method as claimed in claim 9, wherein the splanchnic vein is the hepatic portal vein.

11. A method as claimed in claim 9 or claim 10, wherein the finding of hypertension is measured using the Hepatic Wedge Pressure (HWP), the Free Hepatic Vein Pressure (FHVP), or the Hepatic Vein Pressure Gradient (HVPG).

12. A method of obtaining an indication of a change in pressure in a splanchnic vein of a subject, the method comprising the steps of comparing:
(a) an MRI measurement obtained from the subject's spleen or liver, or a value derived therefrom; and
(b) a corresponding previously-obtained MRI measurement from the subject's spleen or liver, or a corresponding value derived therefrom,
wherein the MRI measurement is T1 mapping or T1 relaxometry or T1 values (ms) or T2* imaging or T2* mapping or T2* values (ms) or T2 mapping or T2 relaxometry or 12 values (ms),
wherein an increase in the measurement or value obtained in step (a) compared to the corresponding measurement or value in step (b) is indicative of an increase in pressure in the splanchnic vein of the subject, and
wherein a decrease in the measurement or value obtained in step (a) compared to the corresponding measurement or value in step (b) is indicative of a decrease in pressure in the splanchnic vein of the subject.

13. A method as claimed in claim 12, wherein the method is for obtaining an indication of a change in pressure in the hepatic portal vein.

14. A method as claimed in claim 12 or claim 13, wherein the pressure is the Hepatic Wedge Pressure (HWP), the Free Hepatic Vein Pressure (FHVP) or the Hepatic Vein Pressure Gradient (HVPG).

15. A method of obtaining an indication of the prognosis of a subject with liver disease or portal hypertension, the method comprising the steps of comparing:
(a) an MRI measurement obtained from the subject's spleen or liver, or a value derived therefrom, with
(b) a corresponding previously-obtained MRI measurement obtained from the subject's spleen or liver, or a corresponding value derived therefrom,
wherein the MRI measurement is T1 mapping or T1 reiaxometry or T1 values (ms) or T2* imaging or T2* mapping or T2* values (ms) or T2 mapping or T2 reiaxometry or 12 values (ms),
wherein an increase in the measurement or value obtained in step (a) compared to the corresponding measurement or value obtained in step (b) is indicative of a decline in the prognosis for the subject,
and
wherein a decrease in the measurement or value obtained in step (a) compared to the corresponding measurement or value in step (b) is indicative of an improvement in the prognosis for the subject.

16. A method of obtaining an indication of the efficacy of a drug which is being used to treat liver disease or portal hypertension in a subject, the method comprising the steps of:
(a) comparing first and second MRI measurements obtained the subject's spleen or liver, or values derived therefrom,
wherein the MRI measurement is T1 mapping or T1 relaxometry or T1 values (ms) or 12* imaging or 12* mapping or 12* values (ms) or 12 mapping or 12 relaxometry or 12 values (ms),
wherein the drug has been administered to the subject in the interval between the taking of the first and second MRI measurements,
wherein an increase in the second measurement or value compared to the first measurement or value is indicative of a lack of efficacy of the drug and
wherein a decrease in the second measurement or value compared to the first measurement or value is indicative of the efficacy of the drug.

17. A method of obtaining an indication of fibrosis in the liver of a subject, the method comprising the steps of correlating:
(a) an MRI measurement obtained from the subject's spleen, or from a value derived therefrom (preferably a value derived from MRI measurements from the subject's spleen and liver), with
(b) the degree of liver fibrosis,
wherein the MRI measurement is T1 mapping or T1 relaxometry or T1 values (ms) or 12* imaging or 12* mapping or T2* values (ms) or 12 mapping or 12 relaxometry or 12 values (ms), thereby obtaining an indication of fibrosis in the liver of the subject.

18. A method as claimed in claim 17, wherein the correlating step comprises correlating the MRI measurement or value obtained from the subject's spleen with corresponding MRI measurements or values obtained from one or more control subjects with defined levels of liver fibrosis.

9. A method of obtaining an indication of fibrosis in the liver of a subject, the method comprising the steps of:
(a) determining from an MRI measurement obtained from the subject's spleen, or from a value derived therefrom, an indication of the degree of liver fibrosis,
wherein the MRI measurement is T1 mapping or T1 relaxometry or T1 values (ms) or T2* imaging or T2* mapping or T2* values (ms) or T2 mapping or T2 relaxometry or T2 values (ms).

20. A method as claimed in claim 19, wherein the determining step comprises correlating the MRI measurement obtained from the subject's spleen, or from a value derived therefrom (preferably a value derived from a MRI measurement of the subject's spleen and liver) with corresponding MRI measurements or values obtained from one or more control subjects with defined levels of liver fibrosis, thereby obtaining an indication of the level of liver fibrosis.

21. A method of obtaining an indication of cirrhosis in the liver of a subject, the method comprising the steps of correlating:
(a) an MRI measurement obtained from the subject's spleen, or a value derived therefrom, with
(b) the severity of liver cirrhosis,
wherein the MRI measurement is T1 mapping or T1 relaxometry or T1 values (ms) or T2* imaging or T2* mapping or T2* values (ms) or T2 mapping or T2 relaxometry or T2 values (ms), thereby obtaining an indication of cirrhosis in the liver of the subject.

22. A method as claimed in claim 21, wherein the correlating step comprises correlating the MRI measurement obtained from the subject's spleen, or a value derived therefrom, with corresponding MRI measurements or values obtained from one or more control subjects with defined severity of liver cirrhosis.

23. A method of obtaining an indication of cirrhosis in the liver of a subject, the method comprising the steps of:
(a) determining from an MRI measurement obtained from the subject's spleen, or a value derived therefrom, an indication of the severity of liver cirrhosis,
wherein the MRI measurement is T1 mapping or T1 relaxometry or T1 values (ms) or T2* imaging or T2* mapping or T2* values (ms) or T2 mapping or T2 relaxometry or T2 values (ms).

24. A method as claimed in claim 23, wherein the determining step comprises correlating the MRI measurement or value obtained from the subject's spleen with corresponding MRI measurements or values obtained from one or more control subjects with defined severity of liver cirrhosis, thereby obtaining an indication of the severity of liver cirrhosis.

25. A method of obtaining an indication of the stage of the liver disease in a subject, the method comprising the step of correlating:
(a) an MRI measurement obtained from the subject's spleen, or a value derived therefrom, with
(b) the stage of the liver disease in the subject,
wherein the MRI measurement is T1 mapping or T1 relaxometry or T1 values (ms) or T2* imaging or 12* mapping or 12* values (ms) or T2 mapping or 12 relaxometry or T2 values (ms).

26. A method as claimed in claim 25, wherein the correlating step comprises correlating the MRI measurement or value obtained from the subject's spleen with corresponding MRI measurements or values obtained from one or more control subjects with defined stages of liver disease.

27. A method of obtaining an indication of the stage of the liver disease in a subject, the method comprising the step of:
(a) determining from an MRI measurement obtained from the subject's spleen, or a value derived therefrom, an indication of the stage of the liver disease in the subject,
wherein the MRI measurement is T1 mapping or T1 relaxometry or T1 values (ms) or T2* imaging or T2* mapping or T2* values (ms) or T2 mapping or T2 relaxometry or T2 values (ms).

28. A method as claimed in claim 27, wherein the determining step comprises correlating the MRI measurement or value obtained from the subject's spleen with corresponding MRI measurements or values obtained from one or more control subjects with defined stages of liver disease, thereby obtaining an indication of the stage of liver disease in the subject.

29. A method as claimed in claim 27 or claim 28, wherein the method provides an indication of the stage of liver fibrosis.

30. A method as claimed in claim 27 or claim 28, wherein the method provides an indication of the stage of liver cirrhosis.

31. A method of obtaining an indication of a change in the stage of the liver disease in a subject, the method comprising the steps of comparing:
(a) an MRI measurement obtained from the subject's spleen, or a value derived therefrom; and
(b) a corresponding previously-obtained MRI measurement or value from the subject's spleen,
wherein the MRI measurement is T1 mapping or T1 relaxometry or T1 values (ms) or T2* imaging or T2* mapping or T2* values (ms) or T2 mapping or T2 relaxometry or T2 values (ms),
wherein an increase in the measurement or value obtained in step (a) compared to the corresponding measurement or value in step (b) is indicative of an increase the stage of the liver disease in the subject,
and
wherein a decrease in the measurement or value obtained in step (a) compared to the corresponding measurement or value in step (b) is indicative of a decrease in the stage of the liver disease in the subject.

32. A method as claimed in claim 31, wherein the method provides an indication of the change in the stage of liver fibrosis.

33. A method as claimed in claim 31, wherein the method provides an indication of the change in the stage of liver cirrhosis.

34. A method of obtaining an indication of the central venous pressure of a subject, the method comprising the steps of correlating:
(a) an MRI measurement obtained from the subject's liver, or a value derived therefrom, with
(b) the central venous pressure,
wherein the MRI measurement is T1 mapping or T1 relaxometry or T1 values (ms) or 12* imaging or 12* mapping or 12* values (ms) or 12 mapping or 12 relaxometry or 12 values (ms), thereby obtaining an indication of the central venous pressure.

35. A method as claimed in claim 34, wherein the correlating step comprises correlating the MRI measurement obtained from the subject's liver, or value derived therefrom, with corresponding MRI measurements, or values derived therefrom, obtained from one or more control subjects with central venous pressure.

36. A method of obtaining an indication of the central venous pressure of a subject, the method comprising the steps of:
(a) determining from an MRI measurement obtained from the subject's liver, or a value derived therefrom, an indication of the central venous pressure,
wherein the MRI measurement is T1 mapping or T1 relaxometry or T1 values (ms) or 12* imaging or 12* mapping or 12* values (ms) or 12 mapping or 12 relaxometry or 12 values (ms).

37. A method as claimed in claim 36, wherein the central venous pressure is the pressure in the hepatic vein, the inferior vena cava or the superior vena cava, or the right atrium.

38. A method of obtaining an indication of the central venous pressure of a subject, the method comprising the steps of:
(a) obtaining an MRI measurement from the subject's liver, or a value derived therefrom; and
(b) correlating the measurement or value obtained in step (a) with the central venous pressure of the subject,
wherein the MRI measurement is T1 mapping or T1 relaxometry or T1 values (ms) or T2* imaging or T2* mapping or T2* values (ms) or T2 mapping or T2 relaxometry or T2 values (ms).

39. A method of obtaining an indication of the central venous pressure of a subject, the method comprising the steps of:
(a) obtaining an MRI measurement from the subject's liver or a value derived therefrom; and
(b) determining from the MRI measurement or value in step (a) an indication of central venous pressure,
wherein the MRI measurement is T1 mapping or T1 relaxometry or T1 values (ms) or T2* imaging or T2* mapping or T2* values (ms) or T2 mapping or T2 relaxometry or T2 values (ms).

40. A method of obtaining an indication of increased central venous pressure in a subject, the method comprising the steps of comparing:
(a) an MRI measurement obtained from the subject's liver, or a value derived therefrom; and
(b) the corresponding measurement or value obtained from a control without increased central venous pressure,
wherein the MRI measurement is T1 mapping or T1 relaxometry or T1 values (ms) or T2* imaging or T2* mapping or T2* values (ms) or T2 mapping or T2 relaxometry or T2 values (ms),
wherein an increase in the measurement or value obtained in step (a) compared to the corresponding measurement or value obtained from the control is indicative of increased central venous pressure in the subject.

41. A method as claimed in claim 40, wherein the increased central venous pressure in the subject is increased pressure in the hepatic vein, the inferior vena cava, the superior vena cava or the right atrium.

42. A method of obtaining an indication of a change in the central venous pressure of a subject, the method comprising the steps of comparing:
(a) an MRI measurement obtained from the subject's liver, or a value derived therefrom; and
(b) a corresponding measurement or value previously-obtained from the subject's liver, wherein the MRI measurement is T1 mapping or T1 relaxometry or T1 values (ms) or T2* imaging or T2* mapping or T2* values (ms) or T2 mapping or T2 relaxometry or T2 values (ms), wherein an increase in the measurement or value obtained in step (a) compared to the corresponding measurement or value in step (b) is indicative of an increase in the central venous pressure of the subject,
and
wherein a decrease in the measurement or value obtained in step (a) compared to the corresponding measurement or value in step (b) is indicative of a decrease in the central venous pressure of the subject.

43. A method as claimed in claim 42, wherein the central venous pressure is the pressure in the hepatic vein, the inferior vena cava, the superior vena cava or the right atrium.

44. A method of obtaining an indication of the prognosis of a subject with heart disease, the method comprising the steps of comparing:
(a) an MRI measurement obtained from the subject's liver, or a value derived therefrom, with
(b) a corresponding previously-obtained MRI measurement or value from the subject's liver,
wherein the MRI measurement is T1 mapping or T1 relaxometry or T1 values (ms) or 12* imaging or 12* mapping or 12* values (ms) or 12 mapping or 12 relaxometry or 12 values (ms),
wherein an increase in the measurement or value obtained in step (a) compared to the corresponding measurement or value in step (b) is indicative of a decline in the prognosis for the subject,
and
wherein a decrease in the measurement obtained in step (a) compared to the corresponding measurement or value in step (b) is indicative of an improvement in the prognosis for the subject.

45. A method of obtaining an indication of the efficacy of a drug which is being used to treat heart disease in a subject, the method comprising the steps of:
(a) comparing first and second MRI measurements from the subject's liver, or values derived therefrom,
wherein the drug has been administered to the subject in the interval between the first and second MRI measurements,
wherein the MRI measurement is T1 mapping or T1 relaxometry or T1 values (ms) or T2* imaging or T2* mapping or T2* values (ms) or T2 mapping or T2 relaxometry or T2 values (ms),
wherein an increase in the second measurement or value compared to the first measurement or value is indicative of a lack of efficacy of the drug
and
wherein a decrease in the second measurement or value compared to the first measurement or value is indicative of the efficacy of the drug.

46. A method as claimed in any one of the preceding claims, wherein the method further comprises the step of correction of the MRI measurement or value for iron overload or deficit from an MRI measurement of iron content obtained from the subject's liver or spleen or from an image of the subject's liver or spleen.

47. A method as claimed in any one of the preceding claims, wherein the measurement is obtained from a MRI image of the liver and/or spleen or the measurement is obtained from a previously-obtained MRI image of the liver and/or spleen.

48. A method as claimed in any one of the preceding claims, wherein the value is derived from MRI measurements obtained or previously-obtained from the liver and/or the spleen or from MRI measurements obtained or previously-obtained from an image of the liver and/or spleen.

49. A method as claimed in claim 48, wherein the value is derived from MRI measurements obtained from the subject's spleen and liver; preferably, wherein the value is derived from or based on the sum of the T1 and/or T2* measurements obtained from the subject's spleen and liver; and more preferably, the value is the sum of the T1 measurements obtained from the subject's spleen and liver.

50. A method as claimed in any one of the preceding claims, wherein the MRI measurement or value derived therefrom which is previously-obtained or from a control subject is obtained from a graph, look-up table, database or mathematical equation.

51. A method as claimed in any one of the preceding claims, wherein the subject is a mammal, preferably a human.

52. A method as claimed in any one of the preceding claims, wherein the subject:
(i) has liver disease, preferably one with liver fibrosis or liver cirrhosis;
(ii) has liver disease and portal hypertension, preferably one with cirrhosis or liver fibrosis;
(iii) has portal hypertension, preferably one with non-cirrhotic portal hypertension or pre-hepatic portal hypertension or hepatic portal hypertension or post hepatic portal hypertension;
(iv) has heart disease, preferably one with right heart failure or congestive heart failure or congenital heart disease or constrictive pericarditis or tricuspid valve disease or valvular heart disease;
(v) has liver disease that is a consequence of heart disease, preferably one with cardiac liver cirrhosis; or
(vi) has increased central venous pressure.

53. A method as claimed in any one of the preceding claims, wherein the method is computer-implemented.

54. A system or apparatus comprising at least one processing means arranged to carry out the steps of a method as claimed in any one of claims 1 to 53.

55. A carrier bearing software comprising instructions for configuring a processor to carry out the steps of a method as claimed in any one of claims 1 to 53.
